(19) United States
(12) Patent Application Publication
(10) Pub. No.: US 2008/0139914 A1
(43) Pub. Date: Jun. 12, 2008

(54) Characterising Body Tissue

(76) Inventors: Matthew Gaved, Cambridge (GB); Michael Farquharson, London (GB)

Correspondence Address: ARENT FOX LLP, 1050 CONNECTICUT AVENUE, N.W., SUITE 400, WASHINGTON, DC, 20036, US

(21) Appl. No.: 10/582,293

(22) PCT Filed: Dec. 13, 2004
(86) PCT No.: PCT/GB04/05185

§ 371 Date: Oct. 5, 2007

(30) Foreign Application Priority Data
Dec. 12, 2003 (GB) 0328870.1
Apr. 23, 2004 (GB) 0409126.0
Nov. 16, 2004 (GB) 0425254.0

(51) Int. Cl. A61B 6/00

(52) U.S. Cl. 600/407

(57) ABSTRACT

The present invention describes a method for analysing body tissue, the method consisting of obtaining data representing a first measured tissue property of a body tissue sample and obtaining data representing a second, different tissue property of the tissue sample, and using the data in combination to provide an analysis of the tissue sample. A method is also described for characterising body tissues as normal or abnormal. The present invention also describes a method for analysing and/or characterising body tissue by obtaining Compton scatter data measured from a body tissue sample on which a penetrating radiation beam is incident and using the data to provide an analysis and/or characterisation of the tissue sample.

### FIELD OF THE INVENTION

The present invention relates to methods for the characterisation of body tissue. More specifically, the invention is concerned with the characterisation of body tissue as normal (e.g. healthy) or abnormal (e.g. pathological). The invention has particular, although not necessarily exclusive, applicability to the diagnosis and management of cancer, including breast cancer.

### BACKGROUND

In order to manage suspected or overt breast cancer, tissue is removed from the patient in the form of a biopsy specimen and subjected to expert analysis by a histopathologist. This information leads to the disease management program for that patient. The analysis requires careful preparation of tissue samples that are then analysed by microscopy for prognostic parameters such as tumour size, type and grade. An important parameter in tissue classification is quantifying the constituent components present in the sample. Interpretation of the histology requires expertise that can only be learnt over many years based on a qualitative analysis of the tissue sample, which is a process prone to intra observer variability.

Despite the relative value of histopathological analysis, there remains a degree of imprecision in predicting tumour behaviour in the individual case. Additional techniques have the potential to fine-tune tissue characterisation to a greater degree than that currently used and hence will improve the targeted management of patients.

In existing research in this field, x-ray fluorescence (XRF) techniques have been used to study trace element composition of breast tissue and have shown that breast cancer is accompanied by changes in trace elements and such measurements could contribute to tissue grading¹. It has also been shown that x-ray diffraction effects can operate as an effective means of distinguishing certain types of tissue^{2,3}. Furthermore, it has been shown that such diffraction effects could be suitably analysed to demonstrate small differences in tissue components and that this analysis could lead to a quantitative characterisation of tissues⁴.

There remains in particular a need for techniques that can be used to characterise tissue to a greater degree in vivo, in order that the need for often painful and distressing biopsies can be reduced.

### SUMMARY OF THE INVENTION

It is one general, preferred aim of the present invention to develop quantitative analytical approaches that could add precision to the characterisation of tissues, in particular to distinguish between normal and diseased (e.g. pathological) tissue.

A preferred aim is to add precision to the several subjective components of tissue analysis, most notably those variables ‘scored’ in breast tumour grading.

In general terms, the invention provides methods for analysing and/or characterising body tissue in which results are obtained by considering a combination of two or more different types of measured tissue properties.

In one aspect, the present invention provides a method analysing body tissue, the method comprising:
obtaining data representing a first measured tissue property of a body tissue sample;
obtaining data representing a second, different tissue property of the tissue sample; and
using the data in combination to provide an analysis of the tissue sample.

In another aspect, the invention provides a method for characterising body tissue, the method comprising:
obtaining data representing a first measured tissue property of a tissue sample;
obtaining data representing a second, different tissue property of the tissue sample; and
using the data in combination to provide a characterisation of the tissue sample.

The characterisation in the second aspect may consist of characterising the tissue sample as normal or abnormal. Alternatively, the characterisation may be performed accounting for many grades of abnormality, for example, on a scale with “Normal” at one end and “Abnormal” at the other, with numerous positions therebetween. As a further alternative, or perhaps additionally, the characterisation may take the form of tissue typing, wherein the characterisation includes an expression of a specific trait, such as the kind of tissue, or the stage of cancer and the like.

In either aspect it is preferred that data representing a third measured tissue property is also used in combination with the other data in the analysis or characterisation of the tissue sample.

It is particularly preferred that data representing four or more measured tissue properties is used in combination in the analysis or characterisation of the tissue sample.

Suitable techniques that can be used to obtain the tissue property data include x-ray fluorescence (XRF), energy or angular dispersive x-ray diffraction (EDXRD), Compton scatter densitometry, low angle x-ray scattering and the measurement of linear attenuation (transmission) coefficients.

The tissues properties that are measured may include the composition of the tissue sample, for instance the presence, concentrations and/or proportions of specific elements or organic compounds. Indeed, a tissue sample may contain more than one type of tissue, for example—fatty or glandular for instance, and the measured property may include information relating to this.

Preferably, in either of the aspects above, the data is used in combination to obtain the desired result by using the data as the input to a predefined calibration model that relates the combined data to one or more tissue characteristics (e.g. normal or abnormal).

In a further aspect, the invention provides a method for creating a tool for the analysis and/or characterisation of body tissue, the method comprising creating a calibration model that relates data representing two or more (preferably three or four or more) measurable tissue properties to one or more tissue characteristics.

The calibration model is preferably produced by using sets of the measured data from tissue samples for which the characteristic(s) (e.g. normal/abnormal) to be determined by the model are already known. These data sets can be used to ‘train’ the model in a known manner.

Other multivariate analysis techniques may be employed.

It is a further general aim of the present invention to provide a method of analysing and/or characterising based on a recognition that Compton scattering densitometry techniques can be used in the analysis of body tissue to very effectively discriminate healthy and abnormal or diseased tissue and to discriminate types of abnormal tissue. Moreover, Compton scattering has been recognised as having potential for application to in vivo tissue characterisation techniques.

The invention of this further general aim provides a method for analysing and/or characterising body tissue, the method comprising:
obtaining Compton scatter data measured from a body tissue sample on which a penetrating (e.g. X-ray) radiation beam is incident; and
using the data to provide an analysis and/or characterisation of the tissue sample.

Compton scatter results from an interaction that occurs between a photon and an electron. For this interaction the electron is assumed to be unbound and acting as a free particle. This assumption can be made if the energy of the incident photon is much greater then the binding energy of the atom. FIG. 8 illustrates the Compton interaction, where E₀ is the energy of the incident photon, E₁ is the energy of the scattered photon, m₀c² is the rest mass energy of the electron and θ is the scattering angle of the photon and φ is the scattering angle of the electron. T is the kinetic energy imparted to the electron.

The electron taking part in the interaction is assumed to be stationary, i.e. the initial energy (Eₑ) and momentum of the electron equals zero. During the interaction the photon imparts some of its energy to the electron. The amount of energy transferred determines the angle of the recoil of the electron and the angle of the resultant photon.

The angle and energy of a Compton scattered particle can be accurately calculated using the principle of conservation of energy and momentum. From FIG. 8 it can be seen that the incident photon has energy E₀=hν and the scattered photon has energy E₁=hν¹. Resolving the energy and momentum into parallel and perpendicular components gives the important Compton scatter equation

$${E}_{1} = \frac{{E}_{0}}{1 + ( \frac{{E}_{0}}{{m}_{0} {c}^{2}} ) ( 1 - cos θ )}$$

hence a measure of Compton scatter can be made by detecting the appropriate energy photons at a given angle.

In some instances it may be sufficient for the Compton scatter data to be as simple as a count of photons detected at a selected angle/energy in a given time period. In other instances, it may be desirable to obtain an absolute measure of electron density (or some other derived measurement). Particularly in the latter case, the Compton scatter data is preferably corrected for attenuation in the tissue sample.

One way to compensate for attenuation effects is to use two radiation sources and two detectors. This is an approach commonly used in bone densitometry, but is less preferable when examining tissue samples, particularly in vivo, because it results in a greater dose of radiation.

A preferred method to correct for attenuation effects is to obtain data representing a measure of the directly transmitted x-ray radiation for each Compton scatter measurement. This data can then be used to correct the Compton scatter data for attenuation in the tissue sample.

Especially at low angles (less than 90°), it is also important to be able to distinguish Compton scatter measurement from the coherent scatter peak. So, where the transmitted radiation is to be used to correct for attenuation it is preferable that the energy of the scattered photons detected is as close as possible to that of the transmitted radiation. This ensures that the attenuation coefficients are not too different for the two measurements. The energy of the incident penetrating radiation beam and the angle selected for Compton scatter measurement are chosen such that the Compton and coherent scatter peaks can be resolved, whilst minimising the separation (i.e. energy) of these peaks. This substantially eliminates self-attenuation effects as it allows one to assume that the attenuation coefficients in the sample affecting both peaks are substantially the same.

Preferably the data is used as the input to a predefined calibration model that relates the Compton scatter data to one or more tissue characteristics (e.g. normal or abnormal or a scale of abnormality having “normal” at one end of the scale and “abnormal” at the other). It is particularly preferred that the Compton scatter data is used as an input to a multivariate model.

Although the specific embodiments describe the various aspects of the invention in relation to breast cancer, it is to be understood that the invention, generally, has a much wider applicability. Indeed, along with analysing and characterising breast tissue for cancer other assessments, such as general nodal assessment, liver, pancreas, prostate, colorectal assessments are contemplated, also urological and gynecological assessments are also envisaged.

### BRIEF DESCRIPTION OF THE DRAWINGS

Embodiments of the invention are described below by way of example with reference to the accompanying drawings, in which:

FIG. 1 is a schematic diagram of EDXRD experimental apparatus employed in the exemplary methods described below according to embodiments of the invention;

FIG. 2 is a series of graphs showing average XRF responses;

FIG. 3 shows EDXRD scatter profiles for normal and diseased tissue;

FIG. 4 shows PLS model predictions for the normal test samples;

FIG. 5 shows PLS model predictions for the diseased test samples;

FIG. 6 shows predictions of tissue type for the normal test samples; and

FIG. 7 shows predictions of tissue type for the disease test samples;

FIG. 8 illustrates the energetics of Compton scattering;

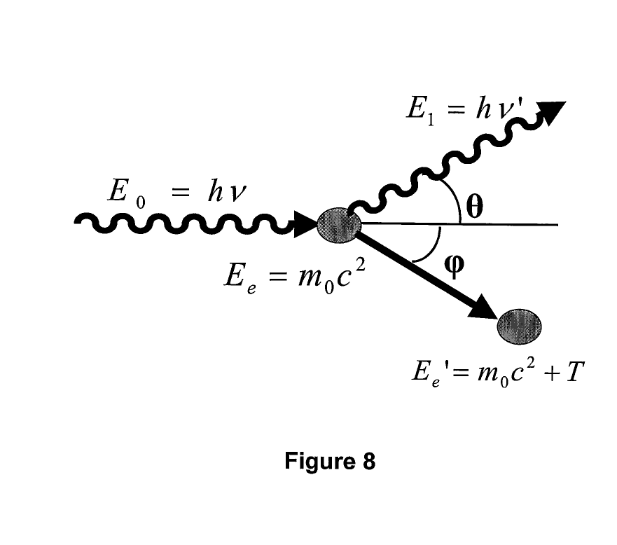

FIG. 9 shows schematically the experimental set-up used for Compton scatter measurements in an example of an embodiment of the invention;

FIG. 10 illustrates the sample holder used in the Compton scatter measurement of the example;

FIG. 11 shows the peak measured with the Ortec GLP-25300 HPGe detector, used in the experiment, using an Am-241 source;

FIG. 12 is a schematic of the electronics used for electron density measurements;

FIG. 13 shows an observed scatter spectrum obtained for one sample during the experiment;

FIG. 14 shows the apparatus of FIG. 2 set-up to take transmission measurements;

FIG. 15 shows a calibration graph for the electron density measurements;

FIG. 16 is a graph of differential scatter coefficient versus theoretical electron density;

FIG. 17 shows the results from the Compton scatter measurements taken from all samples during the experiment;

FIG. 18 is a graph of tabulated tissue values and experimental data.

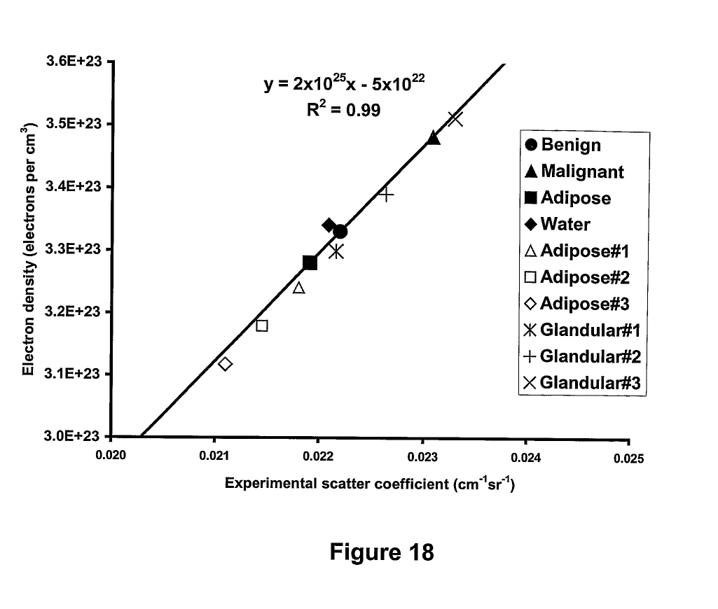

FIG. 19 illustrates the cylindrical geometry used as the sample holder for the measurement of the electron density;

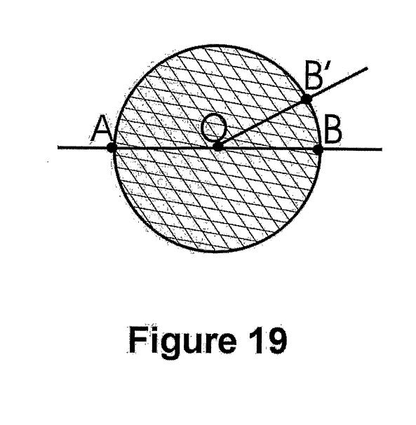

FIG. 20 shows a scatter spectrum from a malignant breast tissue sample;

FIG. 21 shows a calibration graph of the calculated linear scatter coefficients against the counts measured in the Compton scatter peak for the calibration solutions;

FIG. 22 shows a graph of the differential scatter coefficient from experimental data against the calculated electron density for the calibration solutions;

FIG. 23 shows a box plot of the electron density results obtained from the tissue samples; and

FIG. 24 shows a graph of the electron density values for each tissue type.

### DESCRIPTION OF EMBODIMENTS

The following describes a study that has been conducted in vitro using breast tissue samples. The principles exemplified below are, however, more widely applicable, for example to other tissues and in relation to data obtained from in vivo as well as in vitro measurements. The principles can usefully be employed in in vivo imaging applications.

The exemplary embodiments described below employ x-ray fluorescence (XRF) and energy dispersive x-ray diffraction (EDXRD) techniques to reveal the tissue characteristics. The invention is not, however, limited to these two techniques and other techniques may be used in addition or as alternatives to XRD and EDXRD. Other techniques that might be used include Compton scatter densitometry, low angle x-ray scattering and linear attenuation (transmission) coefficients.

As discussed in more detail below, during the study the concentrations of K, Fe, Cu and Zn were measured in 77 breast tissue samples (38 classified as normal and 39 classified as diseased) using X-Ray Fluorescence (XRF) techniques (in other embodiments, concentrations of other elements or organic compounds might be measured). The coherent scattering profiles were also measured using Energy Dispersive X-Ray Diffraction (EDXRD), from which the proportions of adipose and fibrous tissue in the samples were estimated.

The data from 30 normal samples and 30 diseased samples were used as a training set to construct two calibration models, one using a Partial Least Squares (PLS) regression and one using a Principal Component Analysis (PCA) for a Soft Independent Modelling of Class Analogy (SIMCA) technique. The data from the remaining samples, 8 normal and 9 diseased, were presented to each model and predictions were made of the tissue characteristics.

Three data groups were tested: XRF, EDXRD and a combination of both. The XRF data alone proved to be most unreliable indicator of disease state with both types of analysis. The EDXRD data was an improvement, however with both methods of modelling, the ability to predict the tissue type most accurately was by using a combination of the data.

1. Breast Tissue Samples

The tissue samples measured were obtained from mastectomies, lumpectomies and breast reduction surgery. In regard to the latter, a number of healthy breast tissue samples were obtained. The tissue obtained from mastectomies or lumpectomies, was generally taken from the site of a lesion, classified as invasive ductal carcinoma, and in some cases normal tissue was taken from areas distant to the tumours. In line with the available samples, investigations were made for 38 samples classified as normal and 39 samples classified as diseased. The weight of each of the specimens was of the order of 1 g. Most specimens were of thickness in the range of 2-3 mm. Following excision the samples were kept frozen at −85° C., no processing or sample preparation taking place between excision and measurement. For both the XRF and the EDXRD measurements the samples were allowed to thaw before being measured in room temperature.

2. Experimental Procedure

2.1 XRF

The XRF studies were carried out making use of the European Synchrotron Radiation Facility (ESRF), working on the Bending Magnet beamline BM28⁵. Using a simple arrangement of incident synchrotron radiation, tuned to a photon-energy just above the K-absorption edge of interest, particularly low elemental detection limits are achievable (<1 ppm). The high intensities of XRF available allow for short measurement times, providing for a high sample throughput. For the synchrotron photon beam, the plane of polarisation is the same as that of the electron orbit. Thus for a 90° geometry between photons directed on to the sample and the normal to the detector (Si(Li), Gresham Scientific Instruments, Sirius model), the strong linear polarisation of the photon beam provides significant suppression of the scattered photon intensity (fluorescence being unaffected). Given that the detector has lateral extent, the remaining sample-dependent scattered photon (coherent and incoherent) intensity reaching the detector is therefore governed by the solid angle formed between the sample and detector crystal. In addition to providing improvement in the signal to background ratio, control of the scattered radiation intensity allows use of the scattered peak area as a normalisation factor. As tissue is a low Z material, the fact that the detection system cannot resolve the Compton component will not affect the results.

Each element of interest (K, Fe, Cu and Zn) was identified by the photopeak associated with its K_{α} fluorescence photon emission. In order to seek maximum production of the K_{α} photons of interest, the samples were irradiated by photons of energy 500 eV above the particular K absorption edge, being an arrangement which also allows for the resolution of the scattered incident peak and the fluorescence response. The exception to this method was for K, where the data were collected using the same incident photons as that for Fe. In order to quantify the sample concentrations of the elements of interest, calibration curves were constructed for each element.

The calibration standards were aqueous solutions of the elements, the water matrix of the calibration models matching the “wet” nature of the tissue specimens. The following ranges of concentrations were used for the calibrations, as indicative of those expected to be found in tissue:
K: 100, 300 and 1000-4000 ppm in increments of 1000 ppm
Fe: 3-30 ppm in increments of 3 ppm
Cu: 1-10 ppm in increments of 1 ppm
Zn: 2-25 ppm in increments of 2 ppm

The calibration solutions were measured in petri dishes that were sealed with laboratory sealing film (LabSeal, Merck). The tissue specimens were placed on such petri dishes previously filled with purified water and sealed. The specimens were then covered with the same sealing film. The beam size on the specimens was 3 mm×0.5 mm.

The spectra acquired from the standard solutions were analysed using the software PeakFit (PeakFit™ SPSS Inc, AISN Software Inc) developed for spectroscopy. The spectra were smoothed using a procedure based on deconvolution, leading to the removal of peak broadening effects caused by imperfect resolution of the measuring instruments. The spectra were subsequently fitted using a procedure based on the Levenburg-Marquardt non-linear minimisation algorithm. The fitting process took into account a linear baseline resulting in an estimation of the net total of counts integrated over the width of the photopeak. In order to normalise the fluorescence response, the scattered photopeak area was also calculated. The ratio of fluorescence to scattered photon peak area was then used to derive the relationship between element fluorescence and its concentration.

The tissue samples were irradiated under the same conditions as those used for the standard solutions and spectra were collected for each of the elements of interest. The analysis of the spectra also followed the procedure described for the standard solutions. The least squares fit derived from the calibration data, which relates the ratio of fluorescence to scatter photon peak area and the element concentration was used to quantify the levels of each element in each of the samples. It is acknowledged that only a small area of the sample is irradiated but measurements indicate that the inhomogeneity has been found not to significantly alter the profound differences between healthy and cancerous tissues. No correction has been made for matrix effects has been made in this study. However, the interest here is in the comparison between the levels of healthy and cancerous tissue and as any errors are systematic the comparison is not compromised.

2.2 EDXRD

The EDXRD scatter profile of each of the samples was measured using a technique that utilises the scatter of a polyenergetic photon beam at a fixed scatter angle. This technique has been used for a number of biomedical applications, notably that of estimating bone mineral density^{6,7} and more recently for breast tissue analysis². For an overview of applications of X-ray diffraction analysis in crystalline and amorphous body tissue see as an instance Royle et al⁸.

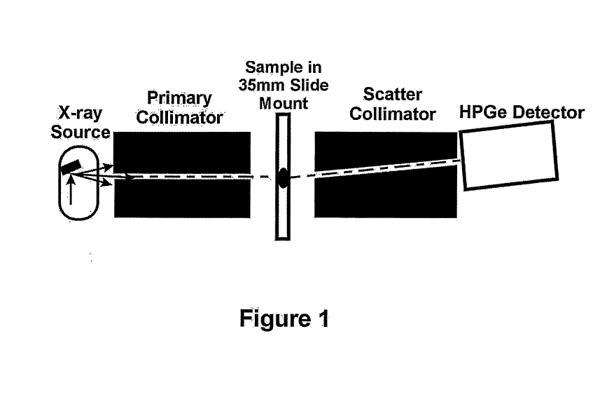

The experimental set up is shown schematically in FIG. 1. A tungsten target x-ray tube operating at 70 kV and 15 mA was used, the intrinsic filtration being 1 mm of beryllium. The incident beam is tightly collimated via a slit cut in a dural slab forming a rectangular cross section of dimensions 1 mm×2 mm. A similar collimation arrangement was set up at a scatter angle of 6° leading to a scattering volume in which the thickness of the sample was enclosed. The samples were mounted in standard 35 mm slide frames, sealed on either side with film 4 microns thick (Ultralene from Glen Spectra Reference Materials). The translator enabled the samples to be moved through the incident beam, in this instance a distance of 3 mm, producing an approximate irradiated volume of 12 mm³. The scattered photons were detected using an HpGe detector (EG&G Ortec). The output pulses were analysed using a multi channel analyser (92× Spectrum Master, EG&G Ortec). The measurement time for each sample was 2400 seconds.

3. Results

3.1 XRF

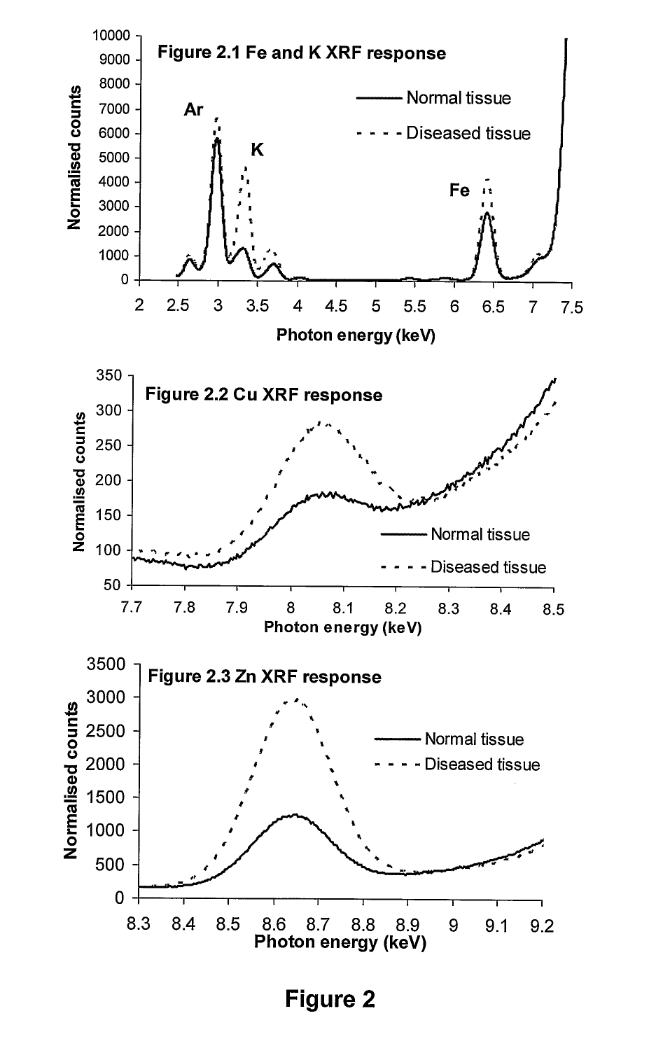

FIG. 2 shows the average of all XRF spectral response for the four elements of interest for normal and diseased tissue. Quantitative values of the elemental concentrations were obtained from the ratio of the XRF response peak to the scattered peak (not shown in figures) via the calibration line.

Table 1 summarises these results for all tissue samples, showing the range of concentrations within a sample group, the mean concentrations and the standard deviation. For details about the measured levels of the elements and the comparison between healthy and cancerous tissue see work by Geraki et al¹.

TABLE 1
Summary of the XRF results for the elements K, Fe, Cu and Zn.

| Units in | K | | Fe | | Cu | | Zn | |
|---|---|---|---|---|---|---|---|---|
| PPM | Normal | Diseased | Normal | Diseased | Normal | Diseased | Normal | Diseased |
| Range | 57-514 | 114-1220 | <D.L.- | <D.L.- | <DL.- | <D.L.- | <D.L.- | <D.L.- |
| | | | 36.64 | 48.98 | 1.53 | 2.09 | 7.51 | 20.19 |
| Mean | 163 | 512 | 8.82 | 17.62 | 0.27 | 0.88 | 2.19 | 6.96 |
| SD | 141 | 336 | 8.74 | 14.09 | 0.22 | 0.52 | 1.37 | 5.31 |

3.2 EDXRD

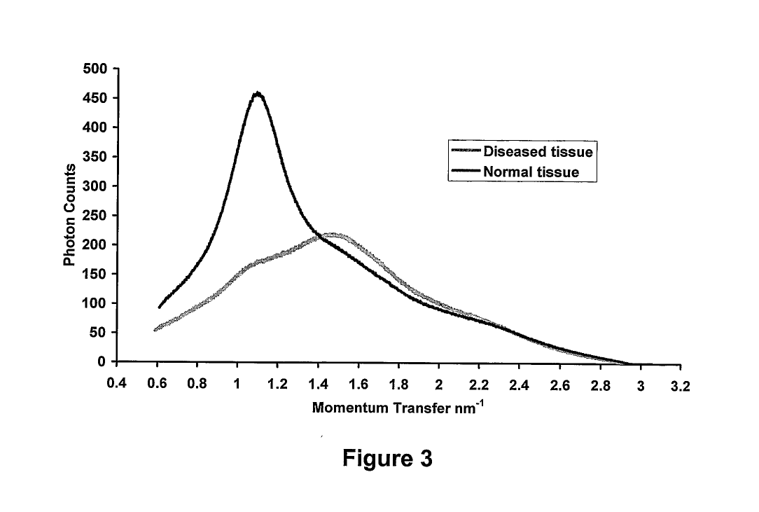

FIG. 3 shows the averaged diffraction spectra for all normal and all diseased tissue samples. The difference in the composition of the two types of specimens is evident.

The characteristic peak from the adipose tissue can be seen at a momentum transfer value of 1.1 nm⁻¹ and the characteristic peak from fibrous tissue is at approximately 1.6 nm⁻¹. These peaks were fitted using the same technique as for the XRF spectra. The evaluated photopeak areas reflecting the presence of adipose and fibrous tissue were then corrected for factors such as the shape of the x-ray tube spectrum and the difference in the attenuation and scatter properties of the two types of tissue. The corrected relative intensities of the two scatter peaks reflect the relative amounts of the two materials in each specimen.

These results show a strong dependence of the relative amounts of the two materials on the type of specimen. The healthy specimens were predominately made of fat (76±9%) while the tumour specimens were mainly composed of fibrous tissue (85±4%).

4. Modelling

The above data were divided into two groups i.e. 30 normal and 30 diseased sample data were used as a training set to produce a calibration model. The data from the remaining samples from each group (8 normal and 9 diseased) were then used as input for the model and the tissue type was predicted.

In the calibration process we use the empirical data (i.e. elemental composition and EDXRD data) and prior knowledge (i.e. quantities known to represent that data) to predict unknown quantitative information from future measurements. In this simple application we are using a multivariate approach i.e. we use many variable measurements X{x(1), x(2) . . . x(n)} to quantify the target variables Y{y(1), y(2) . . . y(m)}. In this case the X variables are the data measured above and the Y variables are the categories of tissue i.e. normal or diseased.

In order to predict the later from the former we need to estimate how X relates to Y and an example of this is a regression model of the form y=Xb+c. The unknown parameters b and c are estimated from the calibration data which can then be used to predict future values of y from measured X.

Another method is to use a classification procedure where models are created that represent a particular classification of variable. This is carried out using principal component analysis on the data belonging to a particular classification. Input data are then analysed and compared with the models and a fit to each classification is established.

There are several statistical calibration methods available for creating a model⁹ and this work uses a partial least squares (PLS) regression and principal component analysis (PCA), which is used for Soft Independent Modelling of Class Analogy (SIMCA) classification. The Unscrambler¹⁰ is a software package that was used to perform multivariate analysis in this study.

4.1 Partial Least Squares (PLS) Regression

Three models were created using the PLS approach. The first used the XRF data alone, the second used the EDXRD data alone and the third used the combined data set. The appropriate data from the test samples (8 normal, 9 diseased) were then presented to the model and predictions of tissue type made.

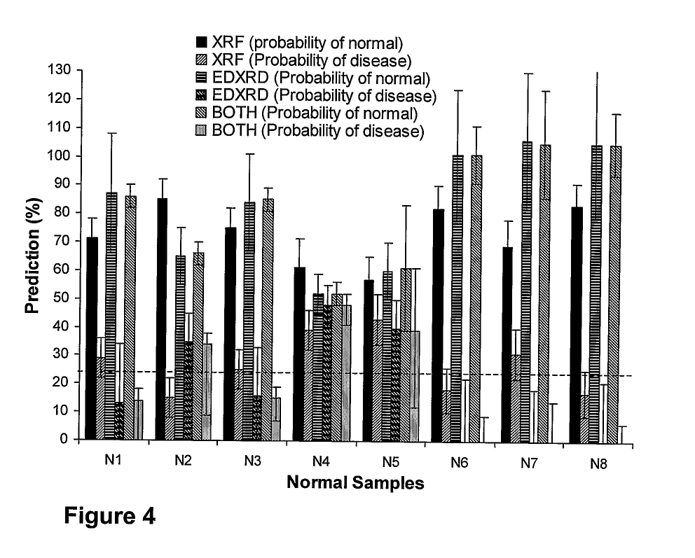

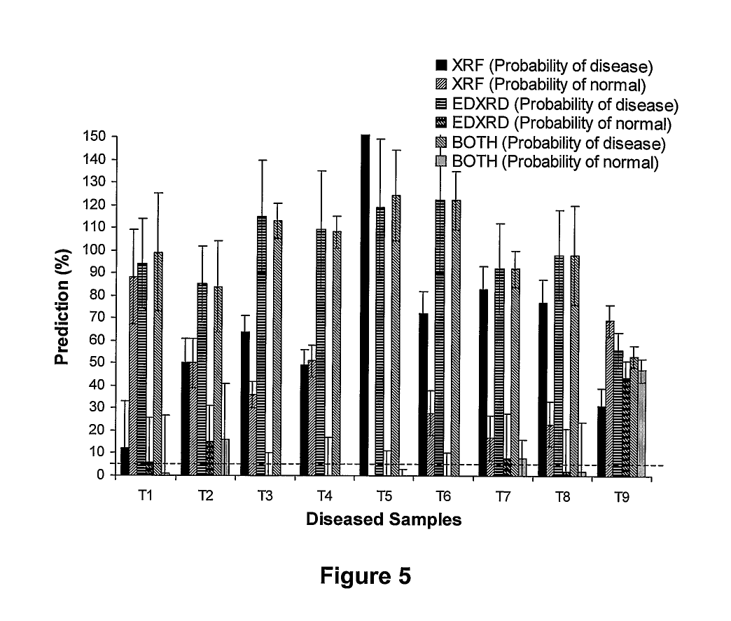

FIGS. 4 and 5 show the predictions for the normal samples for all three models and the predictions for the diseased samples respectively.

4.2 Classification Models

For each of the data groups (XRF, EDXRD and combined data) principal component analysis models were constructed for the healthy and the cancerous samples. The appropriate data from the test samples were presented to the models and a score was obtained indicating how close each sample came to each of the models.

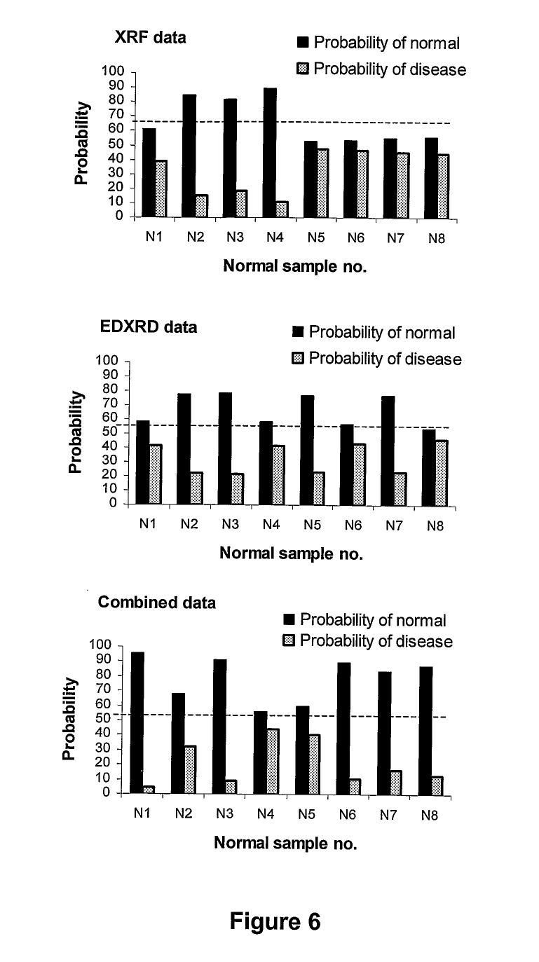

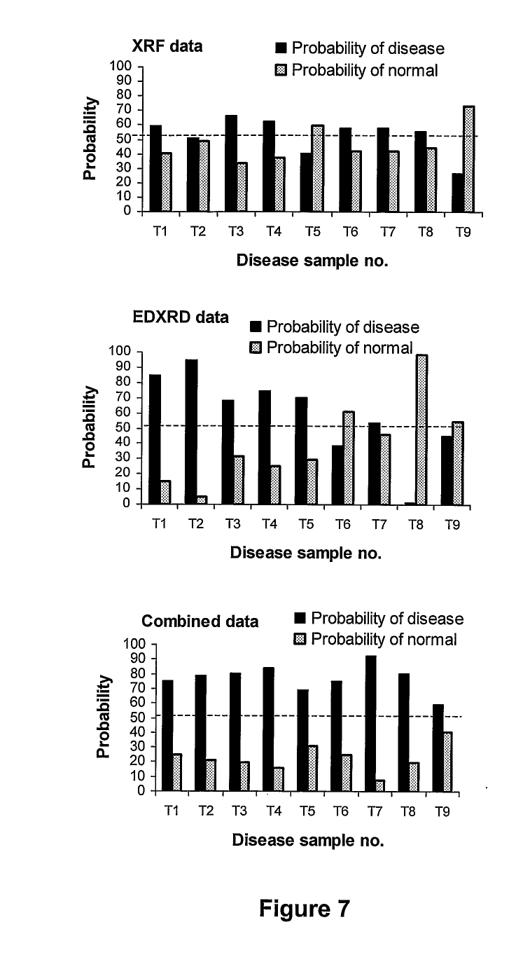

FIGS. 6 and 7 show the predictions for each sample in the normal category and diseased category respectively.

5. Discussion

It can be seen that the XRF model predictions using the PLS approach are the most unreliable with the EDXRD and the combined data being similar. However the uncertainty in the predictions becomes significantly smaller when both data sets are combined.

If an acceptable prediction parameter was chosen at 70% certainty and above, the number of reliable and false predictions can be found. Table 2 summarises the data for the PLS model

TABLE 2
The mean predictions, uncertainty and number of true, false and undecided
predictions for each data group and tissue type using PLS.

| | XRF | | EDXRD | | | |
|---|---|---|---|---|---|---|
| | | Dis- | | Dis- | Combined | |
| | Normal | eased | Normal | eased | Normal | Diseased |
| Mean | 72.8 | 69.8 | 82.2 | 98.5 | 82.6 | 99.2 |
| Prediction | | | | | | |
| (%) | | | | | | |
| Mean | 11.2 | 32.9 | 20.1 | 21.6 | 12.2 | 14.5 |
| Uncertainty | | | | | | |
| (%) | | | | | | |
| True +ve | 3 | 2 | 3 | 6 | 5 | 7 |
| False −ve | 0 | 1 | 0 | 0 | 0 | 0 |
| Undecided | 5 | 6 | 5 | 3 | 3 | 2 |

Similarly, when using the classification technique, the XRF is the most unreliable with an improvement being shown using the EDXRD data. It should be noted that for the normal samples predictions were higher in the normal classification whereas for the diseased samples the wrong classification was made in several instances. The use of the combined data shows a marked improvement in prediction particularly when examining the diseased samples.

As above, choosing a 70% probability cut off limit, the number of accurate predictions can be found. Table 3 summarises the data.

TABLE 3
The mean predictions, true and false positives for each
data group and tissue type using the classification technique.

| | XRF | | | | | |
|---|---|---|---|---|---|---|
| | | Dis- | EDXRD | | Combined | |
| | Normal | eased | Normal | Diseased | Normal | Diseased |
| Mean | 66.8 | 53.2 | 67 | 59 | 77.8 | 77.2 |
| Prediction | | | | | | |
| (%) | | | | | | |
| True +ve | 3 | 0 | 4 | 3 | 6 | 8 |
| False +ve | 0 | 1 | 0 | 1 | 0 | 0 |
| Undecided | 5 | 8 | 4 | 5 | 2 | 1 |

The relative inefficiency of the XRF data (compared to the combined XRF and EDXRD results) in successfully and accurately predicting the type of the test specimens is due to the wide spread of concentrations that characterise the groups of samples, evident by the large associated standard deviations (table 1).

As illustrated by the study described above, embodiments of the present invention can provide improved characterisation of tissue types using a combination of data and an appropriate model. A classification technique has been shown to be particularly successful.

Embodiments of the first general aim of the present invention have been described above by way of example. It will be appreciated that various modifications to that which has been specifically described can be made without departing from the invention. For instance, the study described above to exemplify the invention involved the use of only two types of tissue characterising properties. Other embodiments of the invention may use more than two types of tissue characterising properties or alternative characterising properties. Creating a model using samples that are characterised using a variety of useful parameters may develop useful histopathology tools. Provided the different data groups can represent all the parameters one wishes to quantify, the multivariate approach is a promising method for accurate characterisation of samples.

Although the specific embodiments described above relate primarily to breast cancer, it is to be understood that the invention, generally, has a much wider applicability. Indeed, along with analysing and characterising breast tissue for cancer other assessments, such as general nodal assessment, liver, pancreas, prostate, colorectal assessments are contemplated, also urological and gynecological assessments are also envisaged.

Compton Scattering

The invention of the second general aim is exemplified below with reference to in vitro Compton scatter measurements from uniform samples of body tissue. The general technique is, however, equally applicable to the analysis of non-uniform tissue samples, including in vivo applications.

The experiment was undertaken twice (A & B), each time comprising two sections; Compton scatter measurements were made on all the samples, followed by transmission measurements. This was done in preference to the two measurements being made consecutively for each sample. This method was adopted for two reasons; firstly to ensure consistency of set-up between samples through minimising the moving of equipment and secondly to save time.

Theory

The angle and energy of a Compton scattered particle can be accurately calculated using the principle of conservation of energy and momentum. If the incident photon has energy E₁=hν and the scattered photon has energy E₂=hν¹. Resolving the energy and momentum into parallel and perpendicular components gives the important Compton equation

$${E}_{2} = \frac{{E}_{1}}{1 + ( \frac{{E}_{1}}{{m}_{0} {c}^{2}} ) ( 1 - cos θ )} \qquad ( 1 )$$

where m₀c² is the rest mass energy of the electron and θ is the scattered angle. Consider the cylindrical geometry shown in FIG. 1. A beam of photons is in the direction AB with energy E₁ and a detector is placed at a scatter angle θ to the incident beam. The number of scattered photons, S, with energy E₂ reaching the detector is given by

$$S ∝ ( V {ρ}_{e} ) exp ( - {\int }_{A}^{o} {μ}_{1} ( x ) d x ) exp ( - {\int }_{o}^{{B}^{′}} {μ}_{2} ( x ) d x ) \qquad ( 2 )$$

where V is the volume of scattering material, ρₑ is the electron density of the material in the scattering volume, μ₁ is the attenuation coefficient of photons at the incident energy, and μ₁ is the attenuation coefficient of the Compton photons, scattered through angle θ with reduced energy E₂⁻. If the incident energy E₁ and scatter angle θ are carefully chosen it can be assumed that E₁≈E₂ and therefore that μ₁≈μ₂. Using these assumptions it follows that

$$exp ( - {\int }_{A}^{o} {μ}_{1} ( x ) d x ) exp ( - {\int }_{o}^{{B}^{′}} {μ}_{2} ( x ) d x ) \approx exp ( - {\int }_{A}^{B} {μ}_{1} ( x ) d x ) \qquad ( 3 )$$

and equation (2) becomes

$$S ∝ ( V {ρ}_{e} ) exp ( - {\int }_{A}^{B} {μ}_{1} ( x ) d x ) \qquad ( 4 )$$

From the exponential law of attenuation it is found that

$$\frac{I}{{I}_{0}} = exp ( - \int {μ}_{1} ( x ) d x ) \qquad ( 5 )$$

where I₀ is the incident photon intensity and I is the transmitted photon intensity. Therefore by obtaining a measure of I₀, I and S, the electron density can be found from

$${ρ}_{e} = k ( \frac{S}{T} ) \qquad ( 6 )$$

where S is the scattered count intensity, T=I/I₀ and k is a constant which includes the volume term determined using calibration materials with known electron densities.

### Experiment A

### Method

Ideally a monoenergetic source should be used to ensure that the Compton scatter peak is easily detectable. The characteristic lines produced by the x-ray tube were used to generate a pseudo-monoenergetic source. Using this method the Compton and Coherent peaks can be easily resolved and windowed. The bremsstrahlung background can then be subtracted.

The desired outcome from the experiment was to be able to resolve the Compton and coherent peaks, whilst keeping them as close in energy as possible. The detector characteristics dictate that the minimum resolvable energy is about 1 keV.

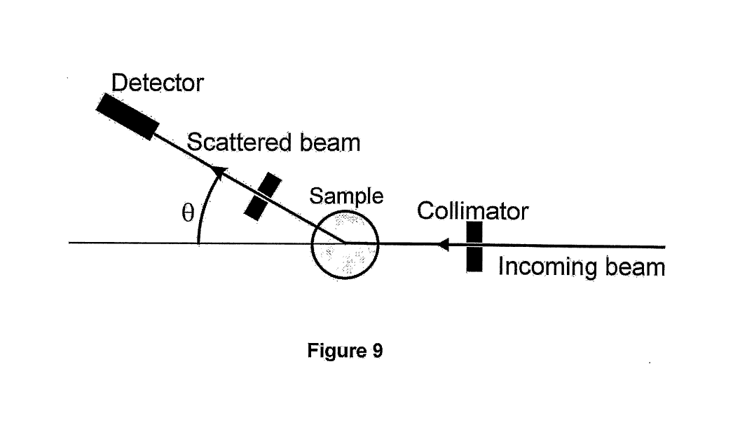

Using the Compton scatter equation set out further above the angle required to give an appropriate difference in energy between the incident and scattered peaks was calculated. To obtain E₀−E′≅1 keV, there was a choice between either using a higher energy and a small scattering angle (defined as θ in FIG. 9 below) or a lower energy with a larger scattering angle. Both of these options were explored. The conclusion was that using a higher energy and minimising the angle offered a number of advantages. Firstly the attenuation of the beam by tissue will be lower with a higher energy. Secondly the scatter is at a maximum in the forward direction and at a minimum at 90°. Therefore the flux reaching the detector will be much higher with a smaller angle, reducing count times considerably. A smaller beam size can also be used, improving the accuracy of the measurement. Larger scattering angles can, however, be used if desired.

The distance between the source, sample and detector were kept to a minimum to decrease the loss of flux due to inverse square law effects. The experimental set-up is shown in FIG. 9.

The incoming x-ray beam was collimated to a 0.5 mm circle, both before and after the sample. This was the smallest beam size obtainable whilst maintaining a reasonable flux. The K_{α} lines from the tungsten target of the x-ray source (E_{kα1}=59.3 keV and E_{kα2}=97 keV) were used. At this energy a scattering angle of 30° would give a peak separation of 1 keV between the Compton and coherent peaks. The exact angle that was set-up in this example was 28.2°. The scattering volume comprises the tissue contained within the intersecting area of the incoming and scattered beam. For this beam collimation and a scattering angle of 28.2° the entire scattering volume was contained within the sample. This means that no air or plastic was contained within the scattering volume. The samples were measured for 20 minutes per position for 12 positions around the sample.

Samples

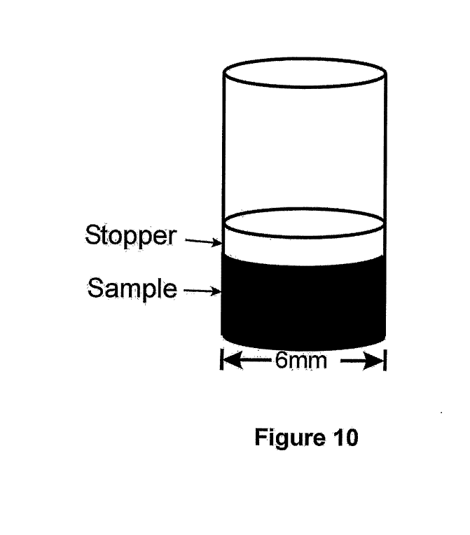

5 samples of each tissue type were chosen for examination. These were 5 fibroadenoma (benign), 5 invasive ductal carcinomas (malignant) and 5 pure adipose (normal) samples. The samples were placed into plastic pots of 6 mm inside diameter and 1 mm wall thickness (illustrated in FIG. 10). Although the walls of the container were relatively thick and would cause significant attenuation of the scattered beam, these containers were chosen because they offered a number of important advantages:
1) The sides were completely rigid so the samples could be placed into the pots and lightly compressed with a stopper (see FIG. 10) to remove any air gaps without the pot becoming distorted. This also minimises tissue movement throughout the experiment.
2) The pots were cheap so each sample could have its own pot for the duration of the experiment, making it possible to move the sample and reposition it accurately
3) The samples also needed to be symmetrical about a centre of rotation.

Equipment

Detector

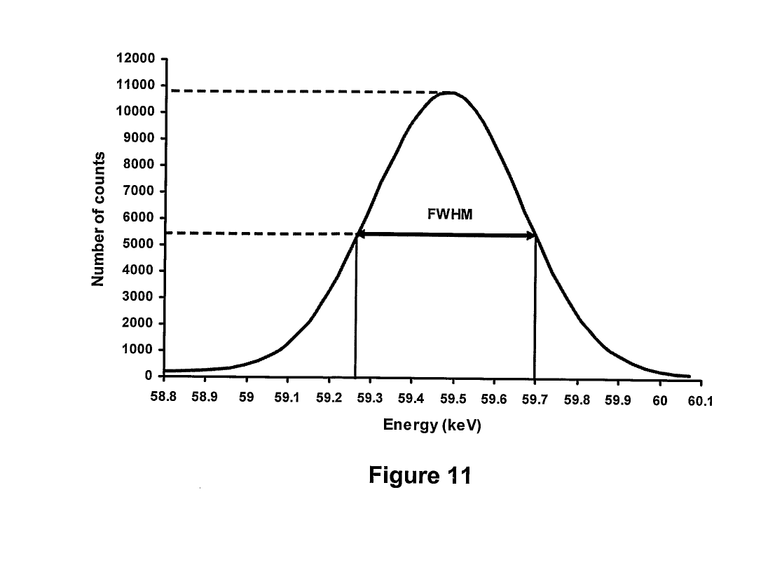

The experiments were performed in the City University Radiation Laboratory using a Pantak HF160 industrial x-ray tube. An HPGe detector was used. This was because a good energy resolution was required for this experiment, to enable the resolution of the Compton and coherent peaks. A peak measured with the Ortec GLP-25300 HPGe detector, which was the detector used throughout all experiments, using an Am-241 source is shown in FIG. 11. The energy resolution is calculated as the FWHM of the peak, as illustrated in FIG. 11. For this detector the energy resolution is 0.435 keV at 59.54 keV (0.73%). The resolution for a Nal(Li) detector at the same energy is about 6-7%. The reason this peak was used to find the energy resolution is because the experiments that were carried out were done using the 57.97 keV K_{α2} peak from tungsten. This is very close in energy to the Am-241 peak at 59.54 keV and so the resolution will be approximately the same.

Electronics

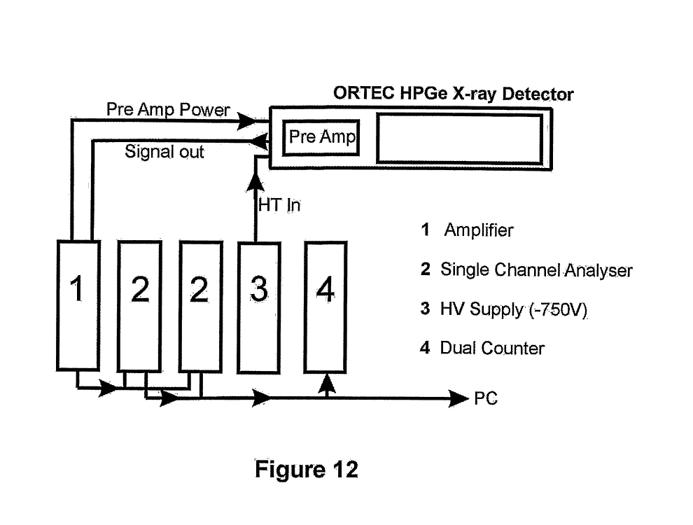

A diagram of the electronics chain is shown in FIG. 12.

The detector was connected via a pre-amp and an amplifier to two single channel analyzers, one to record the Compton peak and one to record a background region. Communication with a PC was enabled via an Ethernet card.

### Windowing

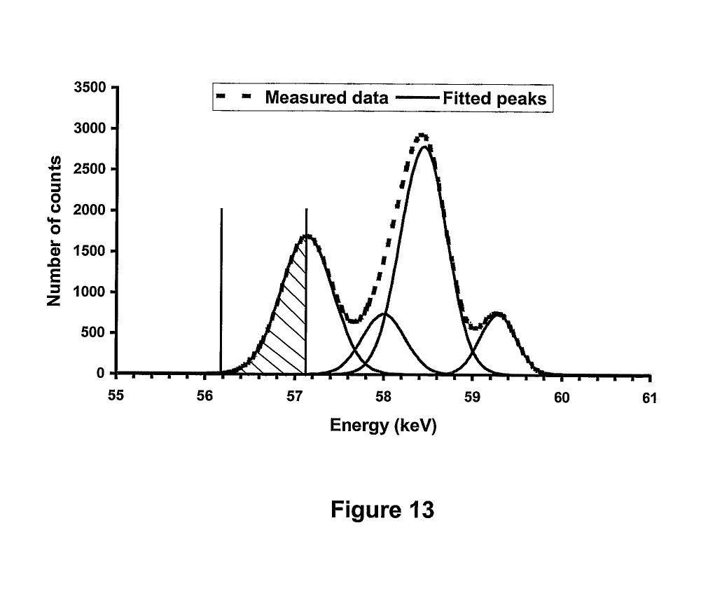

An observed scatter spectrum is shown in FIG. 13. The two coherent peaks of the K_{α1} and K_{α2} W lines can be identified. The two, smaller Compton scatter peaks can be seen.

The K_{α2} Compton peak was measured for this experiment. This is because the K_{α1} peak, although it has a stronger signal, is significantly overlapped by the two coherent peaks.

Transmission Measurements

Method

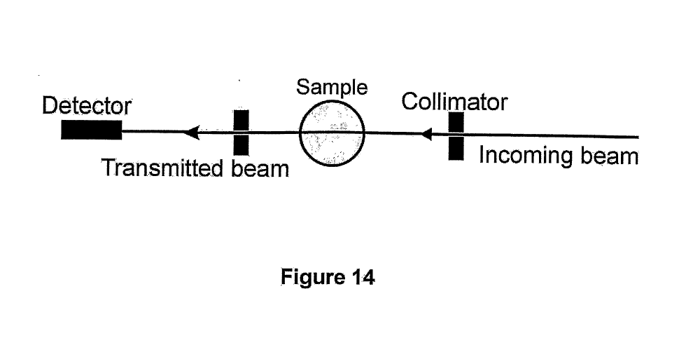

The transmission measurements are a measure of the reduction in intensity of the unscattered peak and are a measure of the loss of counts due to tissue attenuation. For these measurements the detector was placed at zero degrees (See FIG. 14).

System Calibration

As the composition of the tissues being measured is unknown, the electron density measurement system needed to be calibrated. This was done by measuring some substances with a known or calculable electron density. 5 substances were chosen in order to produce a comprehensive calibration graph. The solutions chosen were water, iso-propanol, and solutions of potassium hydrogen phosphate K₂HPO₄. Water and propanol were chosen because they are readily available, easy to handle and have a known electron density that is close to that of tissue. K₂HPO₄ was chosen because it contains elements similar to those found in cellular fluids and so is a good model for human tissue composition. The concentration of the phosphate solutions could easily be varied to provide solutions with differing electron densities. In order to have values close to that of tissue, solutions of 2%, 5% and 10% were used.

In order to verify the scatter data for the calibration solutions the linear differential scattering coefficient can be calculated theoretically as the composition of these solutions is known.

The Klein-Nishina cross-section is dependent on incident photon energy and scattering angle. The Klein-Nishina differential scattering cross section is calculated to be 7.177×10⁻²⁶ cm²/electron for 57.97 keV photons at a 28.2° scattering angle. Using this value and tabulated values for S(x) taken from Hubbell et al (1975) a value for μ_{compton} for each calibration solution was calculated.

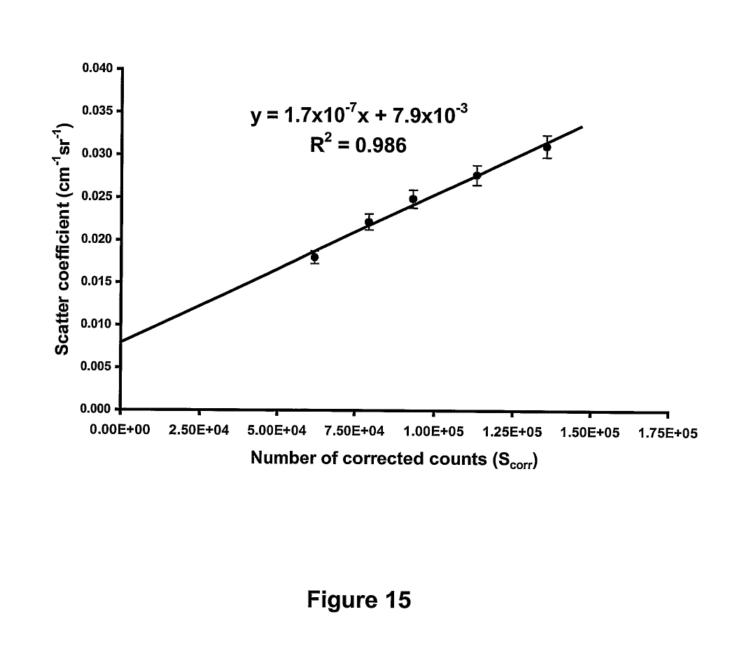

A graph showing the experimental values against the theoretical values is shown in FIG. 15. The corrected scatter counts are the counts measured in the scatter peak corrected for attenuation and are given by

$${S}_{corr} = \frac{[ {S}_{meas} - {B}_{s} ]}{[ ( \frac{{I}_{meas} - {B}_{T}}{{I}_{0} - {B}_{0}} ) ]} \qquad A ( 4.13 )$$

where S_{corr} is the counts recorded in the scatter peak corrected for attenuation. Sₘₑₐₛ is the number of counts in the scatter peak, B_{S} is the background counts in the scatter peak, Iₘₑₐₛ is the number of counts in the transmitted peak, B_{T} is the number of background counts in the transmitted peak, I₀ is the unattenuated count intensity and B₀ is the background area for these counts.

The graph of FIG. 15 can be used to convert the corrected counts measured into differential scatter coefficients for Compton scatter, μ_{S}, where

μ_{S}=k[S_{corr}]+N A(4.14)

In the above equation (4.14) S_{corr} is the corrected scatter counts as described in equation 4.13, N is the systematic experimental error and k is a constant that is found using the calibration graph.

The trend line of the graph does not pass through zero but crosses the y-axis. This suggests that there is a systematic experimental error causing fewer counts to be recorded than expected. This is most likely due to a small amount of copper placed in the beam during the transmission measurements to protect the detector from the high photon flux. The geometry of the set-up was also changed between the scatter and transmission measurements. The detector was moved further away. Due to the inverse square law this would mean that fewer counts would be recorded than expected. These two factors were not corrected for, as they can now be taken into account in this calculation.

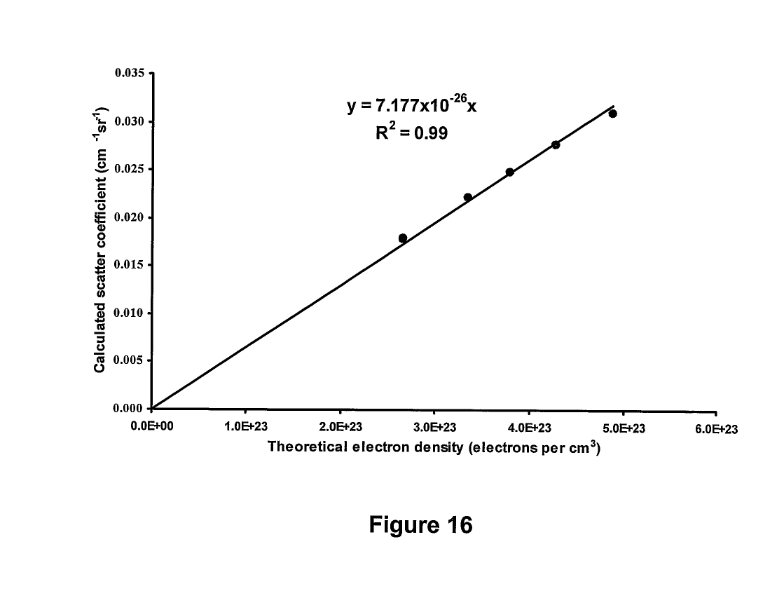

As the composition of the calibration solutions are known the electron densities of these solutions can be calculated using the following formula

$${ρ}_{e} = ρ {N}_{A} \sum \frac{{Z}_{i}}{{A}_{i}} {ϖ}_{i} \qquad A ( 4.15 )$$

where ρ is the physical density of the material and Z/A is the ratio of atomic number to atomic weight for each element of mass fraction ω. Z/A vales are tabulated and were taken from Attix (1996). The graph in FIG. 16 shows the theoretical electron densities plotted against the measured scattering coefficients

For this graph it can be seen that the two quantities correlate almost perfectly with a gradient equal to the Klein-Nishina cross section. This is what is expected as for high values of x the incoherent scattering factors become equal to Z. This agreement confirms the theoretical validity of the experiment.

Results

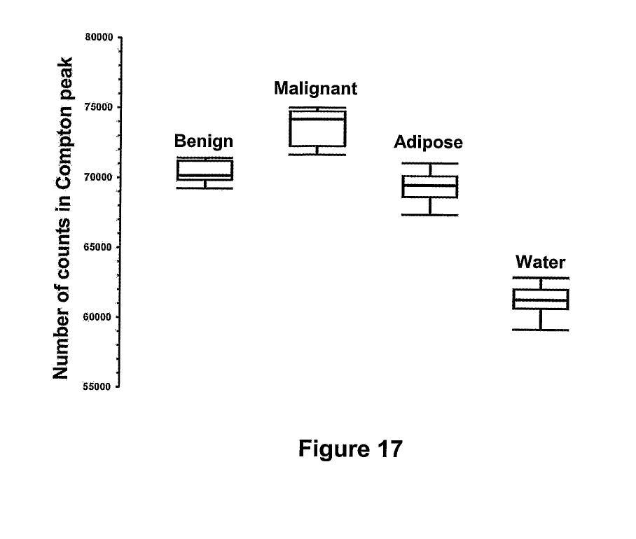

FIG. 17 shows the results that were obtained from the scatter peak measurements.

On the chart in FIG. 17 the median of each tissue type is shown (thick middle line). The interquartile range is contained within the box and the whiskers show the total range.

Analysis

Calculation of Electron Density Values

The graph of FIG. 15 gives a calibration equation to convert the number of counts in the scatter peak into the differential linear scatter coefficient μ_{S}. The equation given by the graph is

μ_{S}=1.737×10⁻⁷x+7.919×10⁻³ A(4.16)

where x is the corrected counts in the Compton peak.

These experimental scatter coefficients are then converted into electron densities using the calibration solution values. This conversion is shown by the trend line in the results graph (FIG. 11).

The results are shown in the graph in FIG. 11. On this graph the values of electron density for standard tissue compositions given in ICRU report 44 (ICRU, 1989) are also displayed. In this report three separate values are given for different tissue compositions. The elemental compositions of these six tissues have been given in the table below. It is worth noting that the values quoted in this report are for healthy tissues only, as there is no published data for malignant tissue growths.

| Tissue | H | C | N | O | Other |
|---|---|---|---|---|---|
| Adipose #1 | 11.2 | 51.7 | 1.3 | 35.5 | 0.1 Na, 0.1 S, 0.1 Cl |
| Adipose #2 | 11.4 | 59.8 | 0.7 | 27.8 | 0.1 Na, 0.1 S, 0.1 Cl |
| Adipose #3 | 11.6 | 68.1 | 0.2 | 19.8 | 0.1 Na, 0.2 S, 0.1 Cl |
| Glandular #1 | 10.9 | 50.6 | 2.3 | 35.8 | 0.1 Na, 0.1 P, 0.1 S, 0.1 Cl |
| Glandular #2 | 10.6 | 33.2 | 3 | 52.7 | 0.1 Na, 0.1 P, 0.2 S, 0.1 Cl |
| Glandular #3 | 10.2 | 15.8 | 3.7 | 69.8 | 0.1 Na, 0.1 P, 0.2 S, 0.1 Cl |

### The Elemental Compositions (Percentage by Mass) of Adult Tissues (ICRU Report 44, 1989)

It is usually assumed that malignant tissue has approximately the same structure as healthy glandular tissue. This is because tumours are usually within fibrous tissue rather than growing in purely fatty (adipose) tissue.

The final results obtained are displayed in the table below.

| Tissue | Electron density (e/cm³) |
|---|---|
| Benign | (3.362 ± 0.141) × 10²³ |
| Malignant | (3.510 ± 0.147) × 10²³ |
| Adipose | (3.312 ± 0.139) × 10²³ |

### Experimental Values Obtained for Tissue Electron Densities

This Difference in measured electron density between tissue types can be used in a model, such as the one described in our co-pending UK patent application GB0328870.1, to determine the tissue type of samples for which the type is unknown. It therefore represents a potentially usefully diagnostic tool. As Compton scatter measurements can also be made in vivo, this approach also potentially lends itself to use as an in vivo, as well as in vitro, diagnostic approach.

Although the three tissue types used to exemplify the invention here are ‘benign’, ‘malignant’ and adipose’, the approach can be applied to the determination of other tissue characteristics or other tissue analysis applications.

### Experiment B

### Material and Methods

Samples

A sample set of four different tissue types were examined comprising of 5 fibroadenoma (benign), 8 invasive ductal carcinomas (malignant), 4 fibrocystic change (non-malignant abnormal) and 5 pure adipose (normal) samples. Each sample was examined at two points. The samples were placed into polythene sample vials of 6 mm inside diameter and 1 mm wall thickness. Although the walls of the vial were relatively thick and would cause some attenuation of the scattered beam, these containers were chosen because they offered a number of important advantages.

The sides were completely rigid so the samples could be placed into a vial and lightly compressed with a stopper without it distorting. This stopper is to remove any air gaps and it also minimises tissue movement throughout the experiment. The containers were cheap so each sample could have its own holder for the duration of the experiment, making it possible to move the sample and reposition it accurately. The samples also needed to be symmetrical about a centre of rotation.

Method

The Kα characteristic lines produced by a tungsten target x-ray tube were utilized as a monoenergetic source to ensure that the Compton scatter peak was detectable. Using this method the Compton and coherent scattered peaks from a recorded spectrum can be easily resolved and windowed and the bremsstrahlung background subtracted. The desired outcome of the experiment was to be able to resolve the Compton and coherent scattered peaks, whilst keeping them as close in energy as possible. The detector characteristics dictated that the minimum resolvable energy is about 1 keV.

The experimental set-up is shown in FIG. 9. The x-ray beam was collimated to 0.5 mm diameter, both before and after the sample. This was the smallest beam size viable whilst maintaining a reasonable flux. The Kα line from the tungsten target of the x-ray source (E_{kα2}=57.97 keV) was used. At this energy a scattering angle of 30° gave a peak separation of 1 keV between the Compton and coherent scatter peaks. The scattering volume comprises of the tissue contained within the intersecting area of the incident and scattered beam. For this beam collimation and scattering angle the entire scattering volume was contained within the sample, with no air or polythene from the vial included. Each sample was measured for a total time of four hours, with the sample being rotated throughout the measurement in order to reduce any errors due to the inhomogeneity of the tissues.

Equipment

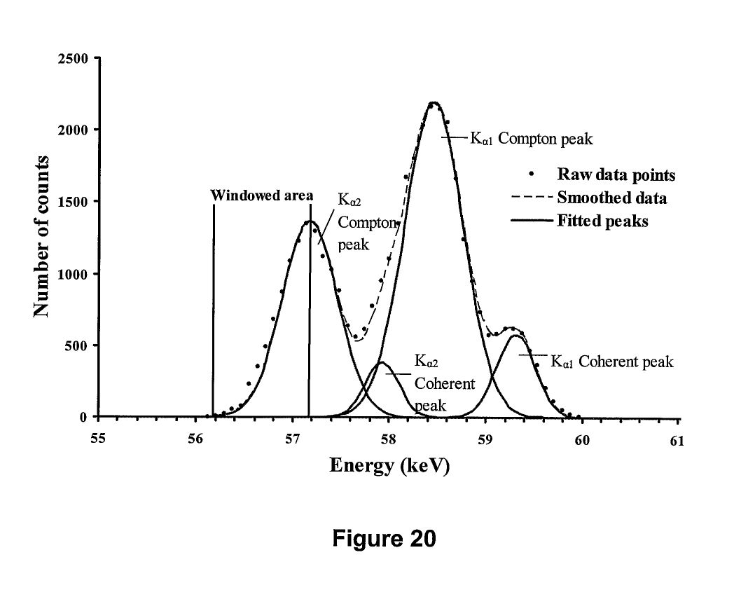

The experiments were performed using a Pantak HF160 industrial x-ray tube. An HPGe detector was used in order to produce the energy resolution required to resolve the Compton and coherent peaks. The energy resolution was measured to be 0.435 keV at 59.54 keV (0.73%). The detector was connected via a pre-amp and an amplifier to two single channel analyzers, one to record the Compton peak and one to record a background region. An observed scatter spectrum of a malignant tissue is shown in FIG. 20.

The two coherent peaks of the K_{α1} and K_{α2} W lines can be identified and the two smaller Compton scatter peaks can be seen. The K_{α2} Compton peak was windowed over an area where there was no superposition of the K_{α2} coherent peak. This windowed area, which was used for the scatter measurements, is also shown in FIG. 20. The transmission measurements for each sample were made by placing the detector at zero degrees and recording the photon intensity with and without a sample in position in the beam.

System Calibration

As the composition of the tissues being measured is unknown, the electron density measurement system needed to be calibrated. This was carried out by measuring substances with a known electron density or one that could be calculated. Five substances were chosen in order to produce a calibration curve.

The solutions chosen were water, iso-propanol, and solutions of potassium hydrogen phosphate K₂HPO₄. Water and propanol were chosen because they are readily available, easy to handle and have a known electron density that is close to that of biological materials. The concentration of the phosphate solutions could be varied to provide solutions with differing electron densities. In order to have values close to that of tissue, solutions of 2%, 5% and 10% were used.

In order to verify the scatter data for the calibration solutions the linear differential scattering coefficient can be calculated theoretically as the composition of these solutions is known. The linear scattering coefficient is a measure of the probability that a photon of incident energy E will be scattered through an angle θ and is given by equation (7):

$$\begin{matrix}{μ}_{compton} = ρ {N}_{A} \frac{S ( x ) d {σ}_{KN}}{M d Ω} & ( 7 ) \\ where \frac{S}{M} = {\sum }_{i} \frac{{S}_{i} ( x )}{{m}_{i}} {ω}_{i} & ( 8 )\end{matrix}$$

where M is the molecular mass of the material, ρ is the mass density; and N_{A} is Avogadro's constant. S(x) is the incoherent scattering factor and the differential scattering cross section is denoted with KN for the Klein-Nishina cross section. The Klein-Nishina differential scattering cross section for the Compton effect is given by

$$\frac{d {σ}_{KN}}{d Ω} = {{r}_{0}^{2} [ \frac{1}{1 + α ( 1 - cos θ )} ]}^{3} [ \frac{1 + cos θ}{2} ] [ 1 + \frac{{{α}^{2} ( 1 - cos θ )}^{2}}{( 1 + {cos}^{2} θ ) [ 1 + α ( 1 - cos θ ) ]} ] \qquad ( 9 )$$

where E is the incident photon energy and θ is the photon scattering angle. α is the ratio of the incident photon energy to the electron rest mass energy given by

$$α = \frac{E}{{m}_{0} {c}^{2}} \qquad ( 10 )$$

and r₀ is the classical electron radius.

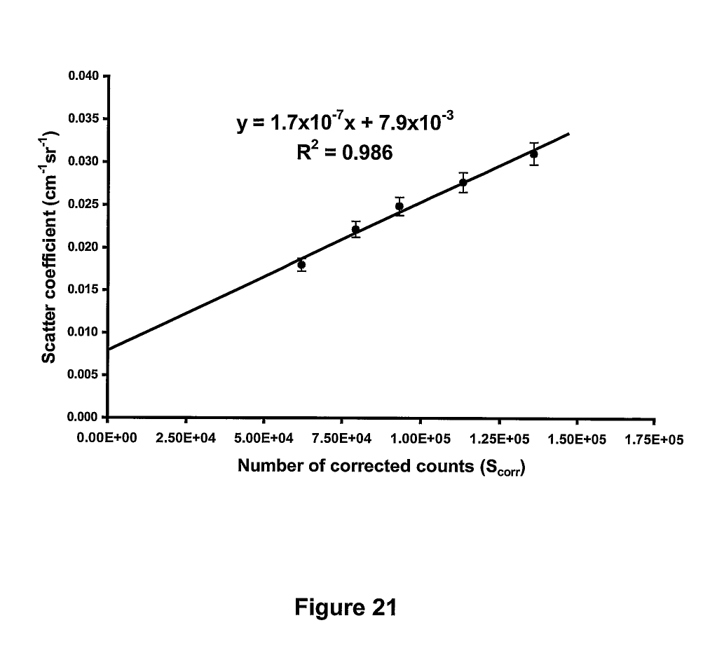

The Klein-Nishina differential scattering cross section is dependent on photon energy and the angle of scatter. It was calculated to be 7.177×10⁻²⁶ cm²/electron for 57.97 keV photons at a 30° scattering angle. Using this value and tabulated values for S(x) taken from Hubbell et al. (1975) a value for μ_{Compton} for each calibration solution was calculated. A graph showing the experimental scatter measurements against the scatter coefficient values calculated from equation (7) is shown in FIG. 21.

The corrected scatter counts are the counts measured in the scatter peak corrected for attenuation and are given by

$${S}_{corr} = \frac{[ {S}_{meas} - {B}_{s} ]}{[ ( \frac{{I}_{meas} - {B}_{T}}{{I}_{0} - {B}_{0}} ) ]} \qquad ( 11 )$$

where S_{corr} is the counts recorded in the scatter peak corrected for attenuation. Sₘₑₐₛ is the number of counts in the scatter peak, B_{S} is the background counts in the scatter peak, Iₘₑₐₛ is the number of counts in the transmitted peak, B_{T} is the number of background counts in the transmitted peak, I₀ is the unattenuated count intensity and B₀ is the background area for these counts. FIG. 21 can be used to convert the corrected counts measured into differential scatter coefficients for Compton scatter, μ_{S}, where

μ_{S}=k[S_{corr}]+N (12)

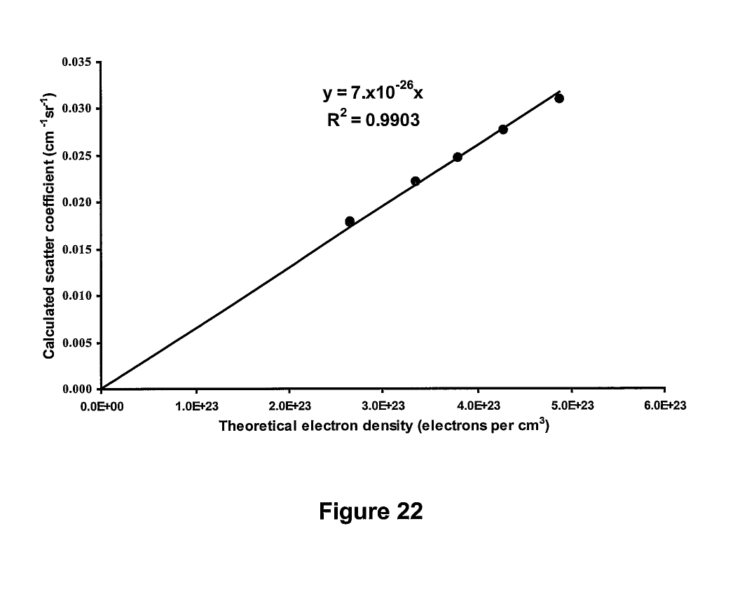

In equation (12) S_{corr} is the corrected scatter counts as described in equation (11), N is the systematic experimental error and k is a constant that is found using the calibration curve. As the composition of the calibration solutions are known the electron densities of these solutions can be calculated using the following formula

$${ρ}_{e} = ρ {N}_{A} \sum \frac{{Z}_{i}}{{A}_{i}} {ϖ}_{i} \qquad ( 13 )$$

where ρ is the physical density of the material and Z/A is the ratio of atomic number to atomic weight for each element of mass fraction ω. Z/A values are tabulated and were taken from (Attix 1986). FIG. 22 shows the theoretical electron densities calculated from equation (13) plotted against the measured scattering coefficients given by equation (12). It can be seen that the two quantities correlate well with a gradient equal to the Klein-Nishina cross section which is expected, as for high values of x the incoherent scattering factors become equal to Z.

Results

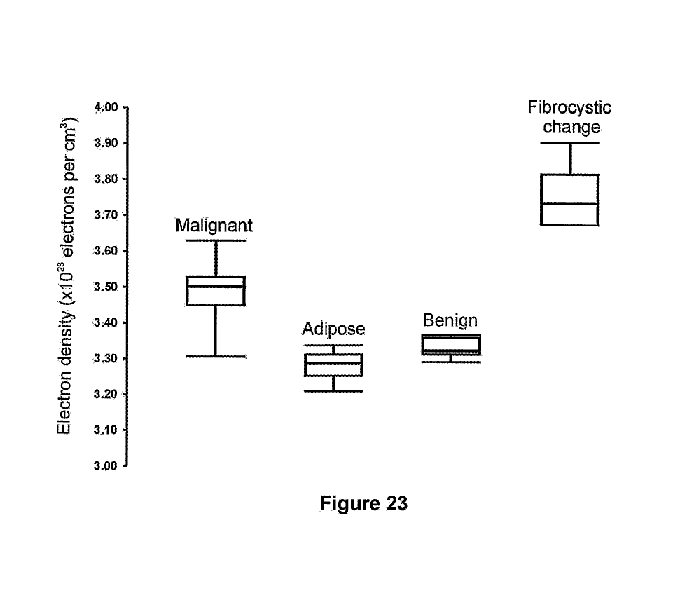

FIG. 23 shows the results of the electron density measurements that were obtained from two points on each sample. The median of each tissue type is shown (thick middle line). The interquartile range is contained within the box and the whiskers show the total range. The graph of FIG. 21 gives a calibration equation to convert the number of counts in the corrected scatter peak into the differential linear scatter coefficient μ_{S}. The equation given by the graph is

μ_{S}=1.737×10⁻⁷x+7.919×10⁻³(14)

where x is the corrected counts in the Compton peak.

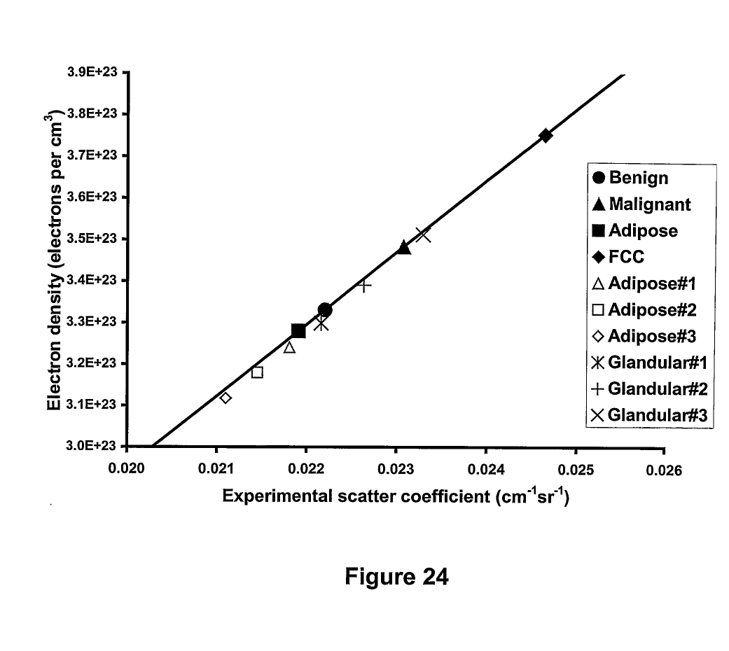

These experimental scatter coefficients are then converted into electron densities using the Klein-Nishina cross section. This conversion is shown by the trend line in FIG. 22. The average results are shown in FIG. 24.

In FIG. 24 the values of electron density for standard tissue compositions given in ICRU report 44 (ICRU 1989) are also displayed. In this report three separate values are given for different tissue compositions. The elemental compositions of these six tissues have been given in table 4. It is worth noting that the values quoted in this report are for healthy tissues only, as there is no published data for malignant tissue growths.

TABLE 4
The elemental compositions (percentage by mass) of
adult tissues (ICRU Report 44, 1989)

| Tissue | H | C | N | O | Other |
|---|---|---|---|---|---|
| Adipose #1 | 11.2 | 51.7 | 1.3 | 35.5 | 0.1 Na, 0.1 S, 0.1 Cl |
| Adipose #2 | 11.4 | 59.8 | 0.7 | 27.8 | 0.1 Na, 0.1 S, 0.1 Cl |
| Adipose #3 | 11.6 | 68.1 | 0.2 | 19.8 | 0.1 Na, 0.2 S, 0.1 Cl |
| Glandular #1 | 10.9 | 50.6 | 2.3 | 35.8 | 0.1 Na, 0.1 P, 0.1 S, 0.1 Cl |
| Glandular #2 | 10.6 | 33.2 | 3 | 52.7 | 0.1 Na, 0.1 P, 0.2 S, 0.1 Cl |
| Glandular #3 | 10.2 | 15.8 | 3.7 | 69.8 | 0.1 Na, 0.1 P, 0.2 S, 0.1 Cl |

It is usually assumed that malignant tissue has approximately the same structure as healthy glandular tissue. This is because tumours are usually within fibrous tissue rather than growing in purely fatty (adipose) tissue.

The final results obtained are displayed in table 5.

TABLE 5
Experimental values obtained for tissue electron densities

| Tissue | Electron density (e/cm³) |
|---|---|
| Benign | (3.330 ± 0.140) × 10²³ |
| Malignant | (3.490 ± 0.147) × 10²³ |
| Adipose | (3.281 ± 0.138) × 10²³ |
| Fibrocystic | (3.752 ± 0.158) × 10²³ |
| change | |

Each individual measurement is subject to statistical variation. The error σ is given as:

$$σ = \sqrt{\frac{\overline{x}}{N}}$$

where x is the mean number of counts if the reading is repeated N times. For the scatter readings each measurement was measured for a sufficient time (4 hours) to ensure that the error on the counts was sufficiently low (<0.5%). Due to time constraints the readings were not repeated.

The largest error is associated with the subtraction of the background counts. The overall error on the background count calculation is 4.2%. This is shown by the error bars in figure 21. Other sources of error are the effect of multiple scatter, the error in positioning and the error in repositioning the sample for the transmission measurements. There is also a widening of the Compton scatter peak caused by the acceptance angle of the pre-detector collimator. None of these other errors have been considered as they are difficult to quantify and are small compared to the background subtraction error outlined above.

Discussion and Conclusions

The results show that there is a detectable difference between the electron density of adipose and malignant tissue, to a value of 6.4%. This difference is consistent with the values found by using the adipose and glandular tissue values from ICRU report 44. The average value for glandular tissue (ICRU44 glandular#2) is 6.2% higher than the average adipose value (ICRU44 adipose #2).

There has been no composition values published for benign (fibroadenoma) or fibrocystic tissues. However the measurements made within this experiment B found a difference in the electron density of benign and malignant tissues to the value of 5.6% and a difference between fibrocystic change and malignant tissue to be 2.3%.

It is difficult to verify these results using the literature as there is no published data from any previous studies using this tissue type. However the high degree of correlation for the calibration solutions (FIG. 21) shows that the system has a reliable accuracy. This inspires confidence in the findings that there is a measurable difference between the benign and malignant tissues.

There is a great deal of evidence to suggest that the metabolism and physiology of tumour cells differ greatly to that of normal and indeed benign cells.

Within a benign tumour growth there is often an increase in cell proliferation but the cells themselves are relatively normal. However, in a malignant lesion the structure and metabolism of the tumour cells and host tissue have a different biochemical structure (Gould 1997). This implies that the increase in the electron density of benign tissues compare to normal may potentially be due to an increase in cell concentration rather than to changes in composition, as seen in malignant tissues. This is consistent with the finding that benign tissues ex-vivo have an electron density which is only slightly higher than normal tissues and malignant tissues display a much larger difference.

Dr Otto Warburg first observed in 1930 that cancer cells have a fundamentally different energy metabolism than normal cells (Warberg 1930). Since then research has shown that tumour cells undergo anaerobic glycolysis, the process where glucose is converted to lactic acid through the process of fermentation. This process is extremely inefficient compared to normal cell aerobic respiration.

Glucose consumption rate has been shown to be proportional to histological grade (Vaupel et al. 1989) and high grade tumours can absorb about 40 times more glucose in order to supply their high energy demands for increased growth. This process is what makes positron emission tomography (PET) imaging so effective at imaging tumours using 18F-FDG, an analogue of glucose. It enables PET to distinguish between benign and malignant neoplasms with a high degree of accuracy, as benign tissues do not exhibit increased glucose consumption (Brock et al. 1997).

Anaerobic glycolysis causes a build up of lactic acid to occur within the tissue. The lactic acid (CH3-CH(OH)—CO(OH)) which builds up within the tumour has a high electron density compared to the host tissue of 8.2×10²³ electrons/cm³ and so could be responsible for the increase in electron density that is measured. There is also an increase of ketones and glutamine (Vaupel et al 1989) which may also increase the overall electron density of tumour tissues. Although no direct measurements have been made of the composition of benign and malignant tissues, the above suggests that there are significant differences in composition. It is difficult to estimate the precise nature of the composition changes, given that there are a number of processes occurring in the tissue during tumorgenesis.

The final tissue type that was examined was fibrocystic change. Although this term encompasses a range of histological changes, the majority are characterised by tissue fibrosis. This is a scarring process whereby the stromal (connective tissue) component of the tissue is increased and collagen accumulates. Although increased mature collagen may be seen in a few other benign disease processes in the breast, the most pronounced increase probably occurs during fibrocystic change. This may account for the finding that this tissue classification had a higher electron density than any other type of tissue, even malignancy. When examining the tissues exhibiting fibrocystic change it is likely that any fluid filled pockets (cysts) will become dispersed during tissue preparation leaving only the dense fibrotic tissue under examination.

Not only is the present invention useful for assessing increased numbers of fibroadenomas, invasive ductal carcinomas and FCC tissues, the present invention may also be adapted to assess healthy fibrous tissue and further disease processes.

### REFERENCES

1. Geraki K, Farquharson M. J. and Bradley D. A. Phys. Med. Biol. 2002 47 (13), 2327-2339
2. Kidane G., Speller R. D., Royle G. R. and Hanby A. M. Phys. Med. Biol. 1999 44 1791-1802
3. Polletti M. E., Goncalves O. D. and Mazzaro I. Phys. Med. Biol. 2002 47 47-63
4. Farquharson M. J., Luggar R. D. and Speller R. D. Appl. Rad. Isot. 1998 48(8) 1075-1082
5. Brown, S. D., Bouchenoire, L., Bowyer, D., Kervin, J., Laundy, D., Longfield, M. J., Mannix, D., Paul, D. F., Stunault, A., Thompson, P., Cooper, M. J., Lucas, C. A. and Stirling, W. G. J. Synchrotron Rad. 2001 8 1172-1181.
6. Royle, G. J. and Speller R. D. Phys. Med. Biol. 1995 40 1487-1498
7. Royle, G. J. and Speller R. D. Phys. Med. Biol. 1991 36 383-389
8. Royle G. J., Farquharson M. J., Speller R. D. and Kidane G. Rad. Phys. Chem 1999 56 247-258
9. Martens H. and Naes T. Multivariate Calibration Wiley, New York 1994
10. CAMO Computer Aided Modelling A/S 1994 The unscrambler version 5.5. Software for multivariate analysis applying PCA, PCR and PLS including experimental design. Olav Tryggvasonsgt. 24, N-7011 Trondheim, Norway.
Al-Bahri J S and Spyrou N M 1998 Electron density of normal and pathological breast tissues using a Compton scattering technique Applied Radiation and Isotopes 49 1677-1684
Attix F 1986 Introduction to radiological physics and radiation dosimetry (New York: Wiley and Sons)
Avtandilov G, Dembo A, Komardin O, Lazarev P, Paukshto M, Shkolnik L, and Zayratiyants O 2000 Human tissue analysis by small-angle X-ray scattering Journal of Applied Crystallography 33 511-514
Brock C S, Meikle S R, and Price P 1997 Does fluorine-18 fluorodeoxyglucose metabolic imaging of tumours benefit oncology? European Journal of Nuclear Medicine 24 691-705
Department of Health. NHS Breast Screening Programme Annual Review. 2003. Report
Duke P R and Hanson J A 1984 Compton Scatter Densitometry with Polychromatic Sources Medical Physics 11 624-632
Evans S H, Bradley D A, Dance D R, Bateman J E, and Jones C H 1991 Measurement of Small-Angle Photon Scattering for Some Breast Tissues and Tissue Substitute Materials Physics in Medicine and Biology 36 7-18
Farquharson M J and Geraki K 2004 The use of combined trace element XRF and EDXRD data as a histopathology tool using a multivariate analysis approach in characterizing breast tissue X-Ray Spectrometry. In press
Fernandez M, Keyrilainen J, Serimaa R, Torkkeli M, Karjalainen-Lindsberg M L, Tenhunen M, Thomlinson W, Urban V, and Suortti P 2002 Small-angle x-ray scattering studies of human breast tissue samples Physics in Medicine and Biology 47 577-592
Geraki K, Farquharson M J, and Bradley D A 2004 X-ray fluorescence and energy dispersive x-ray diffraction for the quantification of elemental concentrations in breast tissue Physics in Medicine and Biology 49 99-110
Gould B 1997 Pathophysiology for the Health Professions (Pennsylvania: W B Saunders)
Hubbell J, Viegele Wm J, Briggs E A, Brown R T, Cromer D T, and Howerton R J 1975 Atomic Form Factors, Incoherent Scattering Functions and Photon Scattering Cross Sections Journal of Physical and Chemical Reference Data 4 471-616
ICRU. Tissue Substitutes in Radiation Dosimetry and Measurement, Report 44. 1989. Maryland, International Commission on Radiation Units and Measurements. Report
Johns P C and Yaffe M J 1983 Coherent Scatter in Diagnostic-Radiology Medical Physics 10 40-50
Kidane G, Speller R D, Royle G J, and Hanby A M 1999 X-ray scatter signatures for normal and neoplastic breast tissues Physics in Medicine and Biology 44 1791-1802
Kosanetzky J, Knoerr B, Harding G, and Neitzel U 1987 X-Ray-Diffraction Measurements of Some Plastic Materials and Body-Tissues Medical Physics 14 526-532
Lewis R A, Rogers K D, Hall C J, Towns-Andrews E, Slawson S, Evans A, Pinder S E, Ellis I O, Boggis C R M, Hufton A P, and Dance D R 2000 Breast cancer diagnosis using scattered X-rays Journal of Synchrotron Radiation 7 348-352
Morin L R M 1982 Molecular-Form Factors and Photon Coherent Scattering Cross-Sections of Water Journal of Physical and Chemical Reference Data 11 1091-1098
Narten A H and Levy H A 1971 Liquid Water: Molecular Correlation Functions from X-Ray Diffraction Journal of Chemical Physics 55 2263-2269
Olkkonen H and Karjalainen P 1975 A Tm-170 gamma scattering technique for the determination of absolute bone density British Journal of Radiology 48 594-597
Peplow D E and Verghese K 1998 Measured molecular coherent scattering form factors of animal tissues, plastics and human breast tissue Physics in Medicine and Biology 43 2431-2452
Poletti M E, Goncalves O D, and Mazzaro I 2002 X-ray scattering from human breast tissues and breast-equivalent materials Physics in Medicine and Biology 47 47-63
Speller R D, Royle G J, and Horrocks J A 1989 Instrumentation and Techniques in Bone-Density Measurement Journal of Physics E-Scientific Instruments 22 202-214
Tartari A, Casnati E, Bonifazzi C, and Baraldi C 1997 Molecular differential cross sections for x-ray coherent scattering in fat and polymethyl methacrylate Physics in Medicine and Biology 42 2551-2560
Vaupel P, Kallinowski F, and Okunieff P 1989 Blood-Flow, Oxygen and Nutrient Supply, and Metabolic Microenvironment of Human-Tumors—A-Review Cancer Research 49 6449-6465
Warberg O 1930 The metabolism of tumours (London: Constable)
Webber C E and Kennet T J 1976 Bone Density Measured by Photon Scattering. I. A System for Clinical Use Physics in Medicine and Biology 21 760-769

1-24. (canceled)

25. A method for characterising body tissue, the method comprising obtaining at least two components of data, wherein data representing a first measured tissue property of a body tissue sample is obtained; data representing a second, different measured tissue property of the tissue sample is obtained;
using the respective data in combination as inputs to a predefined calibration model that relates the combined data to at least one tissue characteristic to provide a characterisation of the tissue sample.

26. A method according to claim 25, wherein the characterisation consists of characterising the tissue sample as normal or abnormal.

27. A method according to claim 25, wherein the characterisation comprises various grades of abnormality.

28. A method according to claim 25, wherein the characterisation comprises tissue typing.

29. A method according to claim 25, wherein the method comprises obtaining at least three components of data representing three different measured tissue properties, the obtained data being used in combination to provide the characterisation of the tissue sample.

30. A method according to claim 25, wherein the method comprises obtaining at least four components of data representing four different measured tissue properties, the obtained data being used in combination to provide the characterisation of the tissue sample.

31. A method according to claim 25, wherein techniques used to obtain the tissue property data include at least one of: x-ray fluorescence (XRF); energy or angular dispersive x-ray diffraction (EDXRD); Compton scatter densitometry; low angle x-ray scattering and the measurement of linear attenuation (transmission) coefficients.

32. A method according to claim 25, wherein the measured tissues properties include the composition of the tissue sample.

33. A method according to claim 25, wherein the method is for characterising body tissue in vitro.

34. A method according to claim 33, wherein the data is obtained from tissue on which substantially no processing and/or no sample preparation has taken place between excision of the tissue and obtaining the data.

35. A method for creating a tool for the characterisation of body tissue, the method comprising creating a calibration model that relates data representing at least two measurable tissue properties to at least one tissue characteristic.

36. A method according to claim 35, wherein the method comprises creating a calibration model that relates data representing at least three measurable tissue properties to at least one tissue characteristic.

37. A method according to claim 35, wherein the method comprises creating a calibration model that relates data representing at least four measurable tissue properties to at least one tissue characteristic.

38. A method according to claim 35, wherein the calibration model is produced by using sets of the measured data from tissue samples for which the or each at least one characteristic to be determined by the model is already known.

39. A method according to claim 35, which comprising the use of a predefined calibration model that relates Compton scatter data to at least one tissue characteristic.
